(19) United States
(12) Patent Application Publication
(10) Pub. No.: US 2024/0320235 A1
(43) Pub. Date: Sep. 26, 2024

(54) DATA PROCESSING METHOD, DATA PROCESSING APPARATUS, AND NON-TRANSITORY COMPUTER-READABLE STORAGE MEDIUM

(71) Applicant: SHIMADZU CORPORATION, Kyoto-shi (JP)

(72) Inventors: Shiori NAGAI, Kyoto-shi (JP); Kenta ADACHI, Kyoto-shi (JP); Satoshi SHIMIZU, Kyoto-shi, (JP); Tomoya TSUDA, Kyoto-shi (JP); Kei AKUTSU, Kyoto-shi (JP)

(21) Appl. No.: 18/586,034

(22) Filed: Feb. 23, 2024

(30) Foreign Application Priority Data
Mar. 20, 2023 (JP) 2023-044138

(51) Int. Cl. G06F 16/25; G06F 16/13

(52) U.S. Cl. CPC G06F 16/254; G06F 16/13

(57) ABSTRACT

A data processing method includes a step for a computer to collect analysis file sets from multiple types of analyzers, the analysis file sets each including analysis data by the analyzer, a step for the computer to store the plurality of collected analysis file sets in a database with the analysis file sets sorted into corresponding collections for each material, and a step for the computer to form a subset by extracting an analysis file set in which a type of the analyzer, preprocessing conditions of a sample measured by the analyzer, and measurement conditions of the sample all match, from the collection.

### CROSS-REFERENCE TO RELATED APPLICATIONS

This application claims priority under 35 U.S.C. § 119 to Japanese Patent Application No. 2023-044138 filed on Mar. 20, 2023, the entire disclosure of which is incorporated herein by reference in its entirety.

### BACKGROUND OF THE INVENTION

### Field of the Invention

The present invention relates to a data processing method, a data processing apparatus, and a non-transitory computer-readable storage medium, and more specifically to a data processing method, a data processing apparatus, and a non-transitory computer-readable storage medium for processing analysis data acquired by multiple types of analyzers.

### Description of the Related Art

The following description sets forth the inventor's knowledge of the related art and problems therein and should not be construed as an admission of knowledge in the prior art.

International Publication No. WO2021/235111 discloses a system for analyzing analysis data acquired by multiple types of analyzers cross-sectionally. In this system, the analysis data acquired by multiple types of analyzers are stored in a database. The data processing apparatus analyzes the analysis data stored in the database using dedicated data analysis software to generate features for use in statistical analyses or AI analyses. The data processing apparatus performs machine learning based on the generated features to build a trained model.

In order to generate features from multiple analysis data stored in a database, it is necessary to transform the analysis data into a format for easier comparison, computation, etc., by performing normalization processing on each piece of the analysis data. The standardization processing includes processing to adjust the scale and the offset of to waveform data, and processing to convert waveform data and image data to quantitative values.

On the other hand, multiple analysis data that differ in at least one of a material of the sample, preprocessing conditions of the sample, the type of the analyzer used for the measurement, and the measurement conditions belong to the database. The multiple analysis data may sometimes include analysis data acquired by measuring samples different in material, under the same preprocessing conditions and the same measurement conditions. For such analysis data, the same standardization processing will make it easier to compare the obtained data with each other.

Further, from the viewpoint of ensuring the accuracy of the analysis data, multiple analysis data belonging to one material sometimes includes analysis data acquired by measuring one sample a plurality of times or analysis data acquired by preparing a plurality of samples from one material and measuring the plurality of samples under the same preprocessing conditions and the same measurement conditions. For such analysis data, it is required to perform the same standardization processing and summarize the acquired data as one feature of the material.

Therefore, in order to perform the standardization processing described above, it is necessary to perform a preliminary task of summarizing analysis data that is worthy of comparison or analysis data that needs to be summarized, i.e., analysis data in which preprocessing conditions of the sample, the type of the analyzer, and the measurement conditions all match. Further, the same standardization processing must be performed on this summarized analysis data group. However, as the types and the number of analysis data stored in the database increase, there is a concern that these works require a great deal of time and effort.

### SUMMARY OF THE INVENTION

The present invention has been made to solve such problems, and the purpose of the present invention is to provide a data processing method, a data processing apparatus, and a non-transitory computer-readable storage medium that are capable of efficiently extracting features from multiple analysis data collected from a plurality of different types of analyzers.

A data processing method according to one aspect of the present invention comprises:
a step for a computer to collect analysis file sets from multiple types of analyzers, the analysis file sets each including analysis data by the analyzer;
a step for the computer to store the plurality of collected analysis file sets in a database with the analysis file sets sorted into corresponding collections for each material; and
a step for the computer to form a subset by extracting an analysis file set in which a type of the analyzer, preprocessing conditions of a sample measured by the analyzer, and measurement conditions of the sample all match, from the collection.

A data processing apparatus according to a second aspect of the present invention is a data processing apparatus capable of communicating with multiple types of analyzers, comprises:
a processor; and
a memory configured to store programs to be executed by the processor.

The processor is configured, according to the programs, to collect analysis file sets from the multiple types of analyzers, the analysis file sets each including analysis data by the analyzer.

The processor is configured is configured to store the plurality of collected analysis file sets in a database with the analysis file sets sorted into corresponding collections for each material.

The processor is configured is configured to form a subset by extracting an analysis file set in which a type of the analyzer, preprocessing conditions of a sample measured by the analyzer, and measurement conditions of the sample all match, from the collection.

A non-transitory computer-readable storage medium according to a third aspect of the present invention stores programs.

The programs make a computer execute
a step of collecting analysis file sets from multiple types of analyzers, the analysis file sets each including analysis data by the analyzer,
a step of storing the plurality of collected analysis file sets in a database with the analysis file sets sorted into corresponding collections for each material, and
a step of forming a subset by extracting an analysis file set in which a type of the analyzer, preprocessing conditions of a sample measured by the analyzer, and measurement conditions of the sample all match, from the collection.

The above and other objects, features, aspects, and advantages of the present invention will become apparent from the following detailed description of the present invention understood in connection with the accompanying drawings.

### BRIEF DESCRIPTION OF THE DRAWINGS

The preferred embodiments of the present disclosure are shown by way of example, and not limitation, in the accompanying figures.

FIG. 1 is a schematic diagram describing a configuration example of an analysis system according to an embodiment of the present invention.

FIG. 2 is a schematic diagram of a hardware configuration example of an information processing apparatus and a data processing apparatus.

FIG. 3 is a schematic diagram of a functional configuration of an information processing apparatus and a data processing apparatus.

FIG. 4 is a diagram for explaining one example of a data structure of an analysis file DB.

FIG. 5 is a diagram of the processing flow in which a data processing apparatus generates a feature table from a plurality of analysis file sets belonging to one collection.

FIG. 6 is a diagram showing one example of a subset table.

FIG. 7 is a diagram showing one example of a subset table to which standardization processing has been performed.

FIG. 8 is a diagram showing one example of a feature table.

FIG. 9 is a flowchart for explaining processing steps performed by a data processing apparatus.

### DETAILED DESCRIPTION OF THE PREFERRED EMBODIMENTS

Hereinafter, some embodiments of the present invention will be described with reference to the attached drawings. Note that, hereinafter, the same or equivalent part in the figures is assigned by the same reference symbol, and the description thereof will not be repeated.

### <Configuration Example of Analysis System>

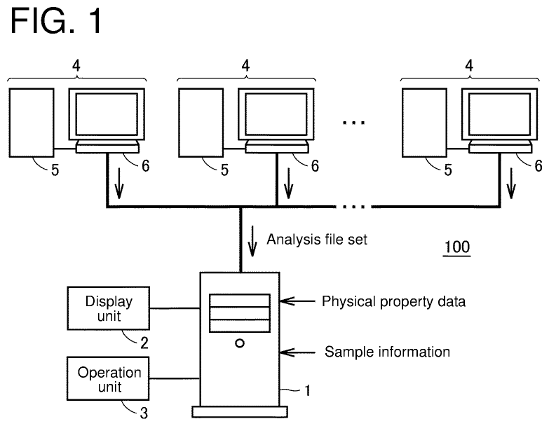

FIG. 1 is a schematic diagram describing a configuration example of an analysis system according to an embodiment of the present invention. The analysis system according to this embodiment can be applied to a system for analyzing analysis data acquired by a plurality of types of analyzers cross-sectionally. As shown in FIG. 1, this analysis system 100 is equipped with a plurality of analyzers 4 and a data processing apparatus 1.

The plurality of analyzers 4 measures a sample. The plurality of analyzers 4 includes multiple types of analyzers. In one aspect, the plurality of analyzers 4 includes a liquid chromatograph (LC), a gas chromatograph (GC), a liquid chromatograph mass spectrometer (LC-MS), a gas chromatograph mass spectrometer (GC-MS), a pyrolysis gas chromatograph mass spectrometer (Py-GC/MS), a scanning electron microscope (SEM), a transmission electron microscope (TEM), an energy dispersive X-ray fluorescence analyzer (EDX), a wavelength dispersive fluorescent X-ray analyzer (WDX), a nuclear magnetic resonator (NMR), a Fourier transform infrared spectrophotometer (FT-IR), etc. The plurality of analyzers 4 may further include a photodiode array detector (LC-PDA), a liquid chromatograph tandem mass spectrometer (LC/MS/MS), a gas chromatograph tandem mass spectrometer (GC/MS/MS), a liquid chromatograph ion trap time-of-flight mass spectrometer (LC/MS-IT-TOF), a near-infrared spectrometer, a tensile tester, a compression testing machine, an emission spectroscopic analyzer (AES), an atomic absorption analyzer (AAS/FL-AAS), a plasma mass spectrometer (ICP-MS), an organic element analyzer, a glow discharge mass spectrometer (GDMS), a particle composition analyzer, a trace total nitrogen automatic analyzer (TN), a high-sensitivity nitrogen carbon analyzer (NC), a thermal analyzer, etc. The analysis system 100 has a plurality of analyzers 4, which makes it possible to perform multifaceted analyses of one sample using a plurality of different types of analysis data.

The analyzer 4 includes a device body 5 and an information processing apparatus 6. The device body 5 measures a sample as an analysis target. To the information processing apparatus 6, identification information on a sample, measurement conditions of the sample, preprocessing conditions of the sample, etc., are input.

The information processing apparatus 6 controls the measurement by the device body 5 according to the input measurement conditions. With this, analysis data based on the measurement results of the sample are acquired. The analysis data may include, for example, electron microscope images acquired by an SEM or a TEM, chromatograms and mass spectra acquired by a GC-MS or an LC-MS, as well as spectra acquired from an FT-IR or an NMR. The information processing apparatus 6 stores the acquired analysis data, together with the identification information of the sample, the measurement conditions, and the preprocessing conditions of the sample, in a data file and saves the data file in its built-in memory. In this specification, this data file is also referred to as “analysis file set.”

The information processing apparatus 6 is connected to the data processing apparatus 1 in a mutually communicable manner. The connection between the information processing apparatus 6 and the data processing apparatus 1 may be wired or wireless. For example, as a communication network connecting the information processing apparatus 6 and the data processing apparatus 1, the Internet can be used. With this, the information processing apparatus 6 of each analyzer 4 can transmit an analysis file set, which is a data file for each sample, to the data processing apparatus 1.

The data processing apparatus 1 is a device for principally managing the analysis data acquired by the plurality of analyzers 4. An analysis file set is input to the data processing apparatus 1 from each of the analyzers 4. It is possible to further input information on the sample (hereinafter also referred to as “sample information”) and physical property data of the sample to the data processing apparatus 1.

The sample information includes identification information to identify the sample (sample ID, sample name, etc.) and information on the sample production (hereinafter also referred to as “recipe data”). The sample recipe data may include, for example, information on the blending quantities of sample raw materials and the sample production process. The physical property data of a sample is data indicating the sample's attributes acquired by means other than the analysis by the analyzer 4.

The data processing apparatus 1 has a built-in database. The database is a storage unit for storing data exchanged between the data processing apparatus 1 and the plurality of analyzers 4, data input from outside the data processing apparatus 1, and data generated in the data processing apparatus 1. The data processing apparatus 1 stores the analysis file set as well as the sample information and the sample's physical property data in a database for each sample, in a linked manner. Note that in the example shown in FIG. 1, it is configured such that the database is built into the data processing apparatus 1, but it can also be configured such that the database is externally attached to the data processing apparatus 1.

### <Hardware Configuration Example of Analysis System>

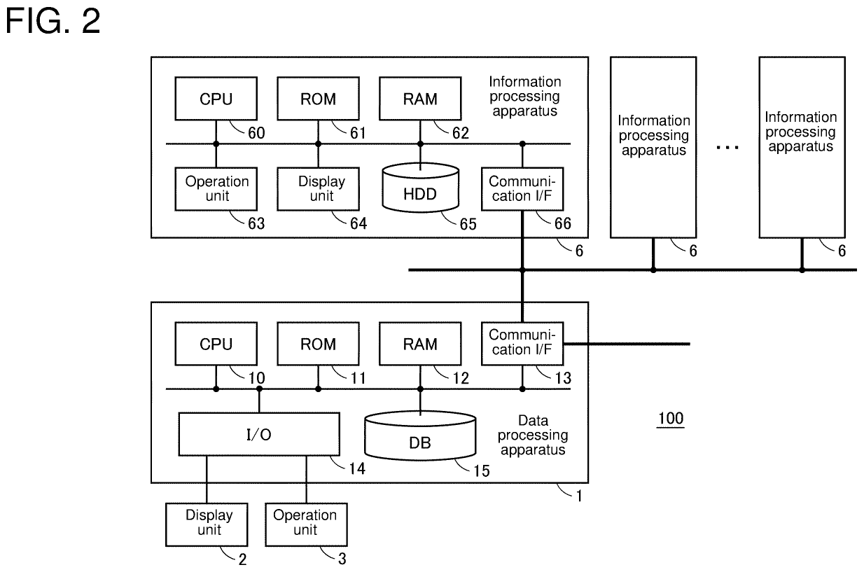

FIG. 2 is a diagram schematically showing a hardware configuration example of the information processing apparatus 6 and the data processing apparatus 1.

### (Hardware Configuration Example of Information Processing Apparatus 6)

As shown in FIG. 2, the information processing apparatus 6 is equipped with a CPU (Central Processing Unit) 60 for controlling the entire analyzer 4 and a storage unit for storing programs and data and is configured to operate according to programs.

The storage unit includes a ROM (Read Only Memory) 61, a RAM (Random Access Memory) 62, and an HDD (Hard Disk Drive) 65. The ROM 61 stores a program to be executed by the CPU 60. The RAM 62 temporarily stores data used during the execution of a program in the CPU 60. The RAM 62 serves as a temporary data memory used as a working area. The HDD 65 is a non-volatile storage device, and stores information, such as, e.g., analysis file sets, generated by the information processing apparatus 6. In addition to or instead of the HDD 65, a semiconductor memory device, such as, e.g., a flash memory, may be used.

The information processing apparatus 6 further includes a communication interface (I/F) 66, an operation unit 63, and a display unit 64. The communication I/F 66 is an interface for the information processing apparatus 6 to communicate with external devices including the device body 5 and the data processing apparatus 1.

The operation unit 63 receives an input including an instruction to the information processing apparatus 6 from the user. The operation unit 63 includes a keyboard, a mouse, and a touch panel integrated with the display screen of the display unit 64 to receive sample measurement conditions and sample identification information.

When setting measurement conditions, the display unit 64 can display, for example, an input screen for the measurement conditions and the sample identification information. During the measurement, the display unit 64 can display the measurement data detected by the device body 5 and the data analysis results by the information processing apparatus 6.

The processing by the analyzer 4 is realized by the respective hardware and the software executed by the CPU 60. In some cases, such software is stored in advance in the ROM 61 or the HDD 65. Further, some software may be distributed as a program product stored in a storage medium, which is not shown in the figure. The software is then read out from the HDD 65 by the CPU 60 and stored in the RAM 62 in a format that can be executed by the CPU 60. The CPU 60 executes this program.

### (Hardware Configuration of Data Processing Apparatus 1)

The data processing apparatus 1 is equipped with a CPU 10 for controlling the entire apparatus and a storage unit for storing programs and data and is configured to operate according to a program. The storage unit includes a ROM 11, a RAM 12, and a database (DB) 15.

The ROM 11 stores programs to be executed by the CPU 10. The RAM 12 temporarily stores data used during the execution of a program in the CPU 10. The RAM 12 functions as a temporary data memory used as a working area.

The DB 15 is a nonvolatile storage device, and stores data exchanged between the data processing apparatus 1 and the plurality of analyzers 4, data input from outside the data processing apparatus 1, and data generated in the data processing apparatus 1. The DB 15 is configured to include an analysis file DB 15A for storing analysis file sets collected from a plurality of analyzers 4 and a feature DB 15B for storing information on features acquired from the plurality of analysis file sets, as described below.

The data processing apparatus 1 further includes a communication I/F 13 and an input/output interface (I/O) 14. The communication I/F 13 is an interface for the data processing apparatus 1 to communicate with external devices including the information processing apparatus 6.

The I/O 14 is an interface for inputs to or outputs from the data processing apparatus 1. The I/O 14 is connected to a display unit 2 and an operation unit 3. When the processing to generate a feature table from a plurality of analysis file sets is executed in the data processing apparatus 1, the display unit 2 can display information on the processing and a user interface screen for receiving user operations.

The operation unit 3 receives inputs including user instructions. The operation unit 3 includes a keyboard and a mouse and receives sample information and sample physical property data. Note that the sample information and the sample physical property data can be received from an external device via the communication I/F 13.

### <Functional Configuration of Analysis System>

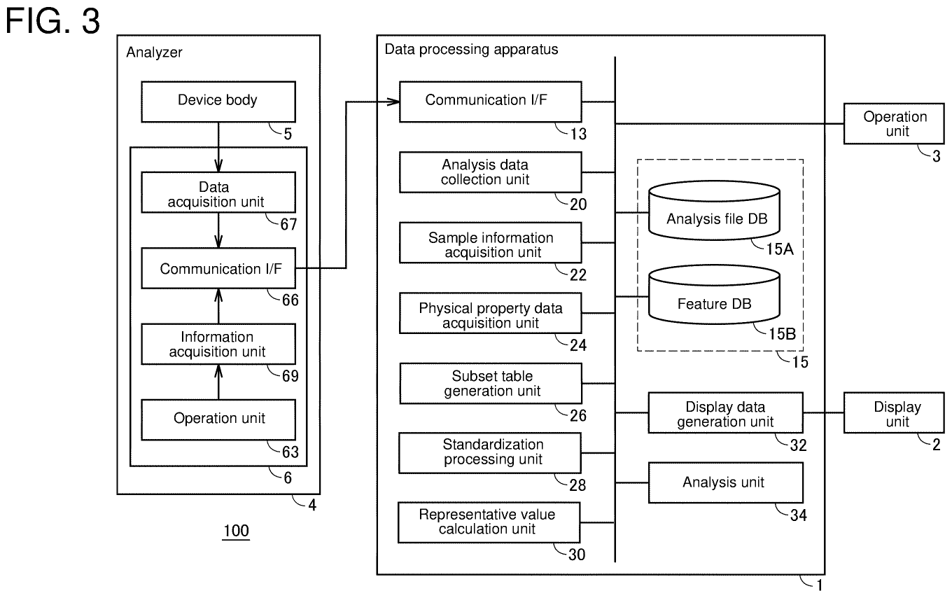

FIG. 3 is a diagram schematically showing a functional configuration of the information processing apparatus 6 and the data processing apparatus 1.

### (Functional Configuration of Information Processing Apparatus 6)

As shown in FIG. 3, the information processing apparatus 6 is configured to include a data acquisition unit 67 and an information acquisition unit 69. These functional configurations are realized by the CPU 60 executing a predetermined program in the information processing apparatus 6 shown in FIG. 2.

The data acquisition unit 67 acquires analysis data based on the measurement results of the sample from the device body 5. For example, in the case where the analyzer 4 is a GC-MS, the analysis data includes chromatograms and mass spectra. In the case where the analyzer 4 is an SEM or a TEM, the analysis data includes image data showing the electron microscope image of the sample. The data acquisition unit 67 transfers the acquired analysis data to the communication I/F 66.

The information acquisition unit 69 acquires the information received by the operation unit 63. Specifically, the information acquisition unit 69 acquires information indicating sample identification information, sample measurement conditions, and sample preprocessing conditions. The sample identification information includes, for example, the sample name, the product name, the model number, and the serial number of the product to be sampled. The sample measurement conditions include device parameters including the name and the model number of the analyzer to be used, and measurement parameters indicating the measurement conditions, such as, e.g., voltage and/or current application conditions and temperature conditions. Preprocessing of a sample means processing of a sample to be performed prior to performing an analysis or a measurement of the sample so that the sample becomes a state suitable for the analysis. The preprocessing of a sample includes, for example, filtration processing and grinding processing to remove unwanted components, ashing to decompose and remove organic matter from a sample, and solid-phase extraction processing performed prior to a liquid chromatography analysis.

The communication I/F 66 transmits an analysis file set in which the acquired analysis data, measurement conditions, and sample identification information are combined into one file to the data processing apparatus 1.

### (Functional Configuration of Data Processing Apparatus 1)

The data processing apparatus 1 is equipped with an analysis data collection unit 20, a sample information acquisition unit 22, a physical property data acquisition unit 24, a subset table generation unit 26, a standardization processing unit 28, a representative value calculation unit 30, a display data generation unit 32, an analysis unit 34, an analysis file DB 15A, and a feature DB 15B. These functional configurations are realized by the CPU 10 executing a predetermined program in the data processing apparatus 1 shown in FIG. 2.

The analysis data collection unit 20 collects an analysis file set transmitted from the information processing apparatus 6 of each analyzer 4 via the communication I/F 13. The analysis file set includes analysis data of a sample, identification information of a sample, measurement conditions, and preprocessing conditions of a sample. The analysis data collection unit 20 stores the collected analysis file sets in the analysis file DB 15A. The analysis file DB 15A stores a wide variety of analysis file sets collected from the plurality of analyzers 4.

The sample information acquisition unit 22 acquires the sample information received by the operation unit 3. The sample information includes the sample identification information (sample ID, sample name, etc.) and the sample recipe data.

The physical property data acquisition unit 24 acquires the physical property data of the sample received by the operation unit 3. The physical property data of a sample is data indicating the sample's attributes, including, for example, a value indicating the sample's performance and a value indicating the deterioration degree of the sample.

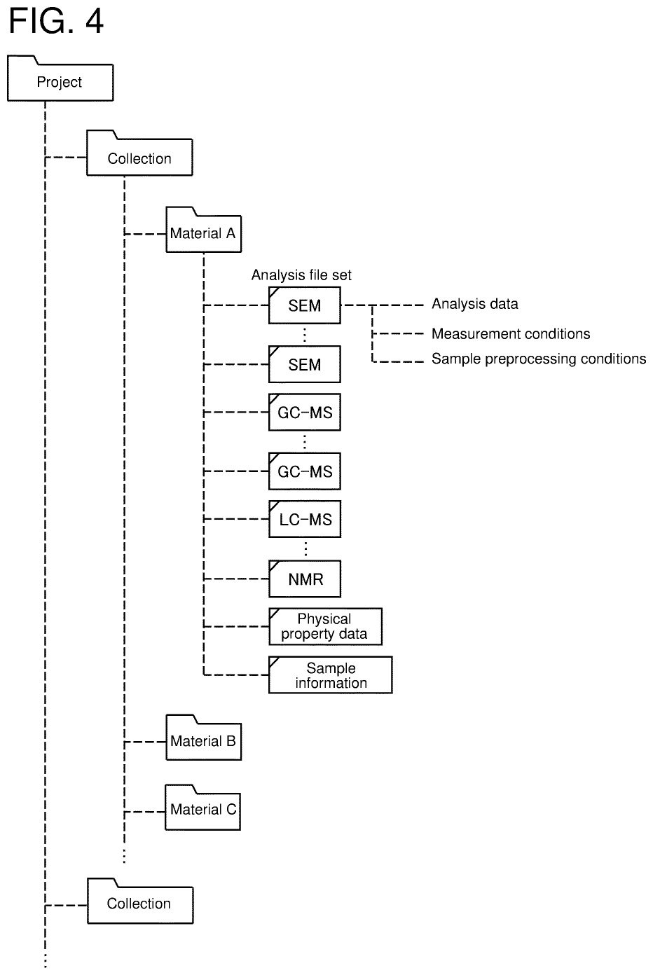

In the analysis file DB 15A, the analysis file sets collected by the analysis data collection unit 20, and the sample information and the physical property data acquired by the sample information acquisition unit 22 and the physical property data acquisition unit 24 are stored in a linked manner. FIG. 4 is a diagram for explaining one example of the data structure of the analysis file DB 15A. As shown in FIG. 4, in the analysis file DB 15A, a plurality of analysis file sets is stored in a frame called “Project.” The term “Project” means a frame that defines a collection of materials to be controlled to achieve a common goal, such as, e.g., a development of a new product. In this project, a plurality of analysis file sets is grouped together and stored by collection. The “collection” means a collection of materials to be used for AI analyses, statistical analyses, etc.

For example, in the case where the project is a development of a secondary battery, such as, e.g., a lithium-ion battery, the project generates a collection on positive electrode materials for a secondary battery, a collection on negative electrode materials, a collection on electrolytes, and so on. Then, a plurality of materials serving as positive electrode materials are grouped together in a collection related to a positive electrode material, a plurality of materials serving as negative electrode materials are grouped together in a collection related to a negative electrode material, and a plurality of materials serving as electrolytes are grouped together in a collection related to an electrolyte.

In each collection, the plurality of analysis file sets is sorted and stored for each material. Which material each analysis file set is sorted into can be determined from the sample identification information included in the analysis file set or from the sample information associated with the analysis file set. With this, as shown in FIG. 4, a plurality of analysis file sets collected from multiple types of analyzers 4 is stored for one material. Sample information and physical property data are further stored for one material.

Here, in order to use the plurality of analysis file sets stored in the analysis file DB 15A for machine learning, it is necessary to transform the analysis data included in each analysis file set into a form for easier comparison and calculation by performing standardization processing on the analysis data included in each analysis file set. The term “standardization processing” as used in this specification refers to the standardizing the analysis data into a form for easir comparison. The standardization processing includes processing to adjust the scale and the offset of the waveform data, as well as processing to convert the waveform data and the image data to quantitative values.

The standardization processing includes, for example, alignment processing to correct the retention time discrepancy so that the total ion chromatograms (TIC) acquired from a GC-MS can be easily compared with each other. This alignment processing allows the retention times of a plurality of TICs to be aligned.

Further, the standardization processing includes processing to acquire quantitative values from analysis data by analyzing the analysis data with dedicated data analysis software, such as processing to calculate a peak area and a peak intensity from chromatograms acquired from a GC-MS and processing to calculate a particle area and a particle diameter from electron microscope images acquired from an SEM.

The analysis data subjected to standardization processing are used in machine learning as features that represent the characteristics of the analysis data. The feature data include, for example, electron microscope images acquired by an SEM or a TEM, chromatograms acquired by a GC-MS or an LC-MS, analysis data such as spectra acquired by an FT-IR or an NMR, and sample composition, concentration, molecular structure, number of molecules, molecular weight, degree of polymerization, particle diameter, particle area, number of particles, particle dispersion, peak intensity, peak area, peak slope, compound concentration, compound amount, absorbance, reflectance, transmittance, sample test intensity, Young's modulus, tensile strength, deformation amount, strain amount, fracture time, average interparticle distance, dielectric dissipation factor, elongation, spring strength, loss factor, glass dislocation temperature, and thermal expansion coefficient.

On the other hand, as shown in FIG. 4, one collection belongs to multiple analysis data that differ in at least one of the following aspects: a material, sample preprocessing conditions, type of analyzer used for the measurement (instrument type), and measurement conditions. The multiple analysis data may sometimes include analysis data acquired by measuring samples different in material, under the same preprocessing conditions and the same measurement conditions. For such analysis data, the same standardization processing will make it easier to compare the obtained data with each other.

Further, multiple analysis data belonging to one material sometimes includes analysis data acquired by measuring one sample a plurality of times under the same measurement conditions or analysis data acquired by preparing a plurality of samples from one material and measuring the plurality of samples under the same preprocessing conditions and the same measurement conditions, from the viewpoint of ensuring the accuracy of the analysis data. For such analysis data, it is required to perform the same standardization processing and summarize the acquired data into one feature of the material.

Therefore, in order to perform the standardization processing described above, it is necessary to perform a preliminary work to summarize analysis data worth comparing or analysis data that need to be summarized, i.e., analysis data in which sample preprocessing conditions, apparatus type, and measurement conditions all match. Further, this summarized analysis data group requires the same standardization processing. However, as the types and the number of analysis data stored in the database increase, there is a concern that these works require much time and effort.

To address these concerns, in this embodiment, the data processing apparatus 1 generates subset tables from the plurality of analysis file sets stored in the analysis file DB 15A as a preprocessing step for generating the feature table. The “subset table” as used in this specification is a table format showing a subset configured by taking out a portion of a plurality of analysis file sets belonging to one collection (hereinafter referred to as a “subset”). The data processing apparatus 1 performs batch normalization processing in a subset table unit and then generates a feature table using the data generated by the normalization processing.

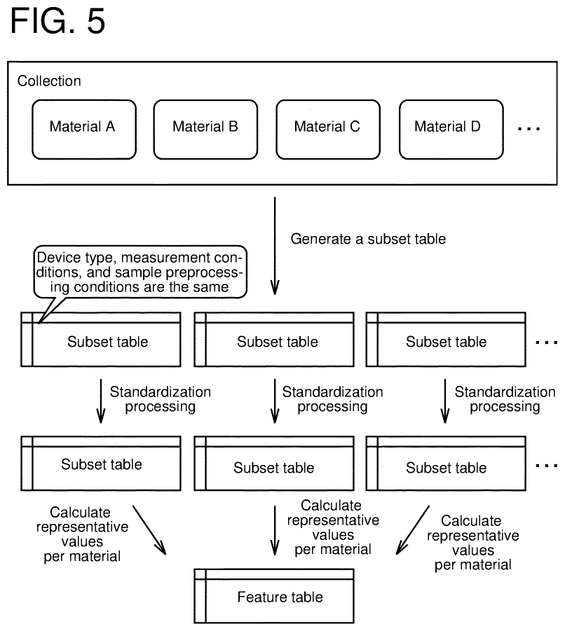

FIG. 5 shows the processing flow in which the data processing apparatus 1 generates a feature table from a plurality of analysis file sets belonging to one collection.

Referring to FIG. 5, initially, the subset table generation unit 26 extracts analysis file sets that all match the analyzer type, preprocessing conditions of the sample, and measurement conditions, from a plurality of analysis file sets belonging to one collection and forms a subset. The device type, the preprocessing conditions of the sample, and the measurement conditions can be specified by the user using the operation unit 3. The subset table generation unit 26 extracts analysis file sets that match the conditions specified by the user, based on the sample identification information, the sample measurement conditions, and the preprocessing conditions of the sample included in each analysis file set.

The formed subset corresponds to an analysis file set group with analysis data to be compared or summarized with the same standardization processing. Note that the subset is formed over material differences within one collection. However, an analysis file set belonging to another collection is not included in the subset.

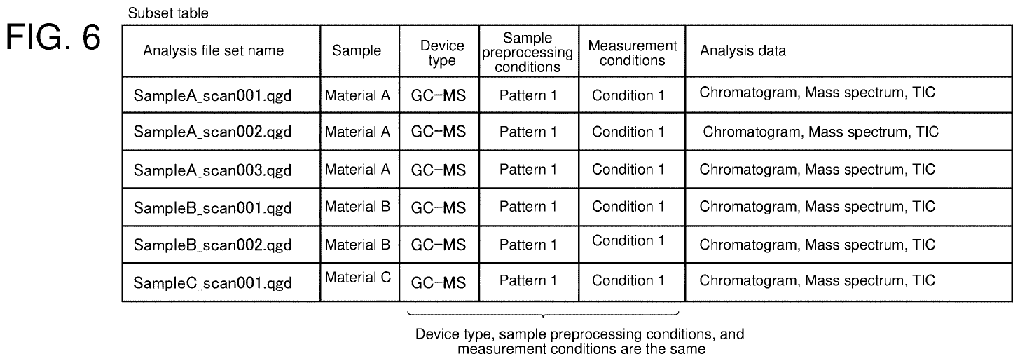

Next, the subset table generation unit 26 generates subset tables. FIG. 6 is a diagram showing one example of a subset table. As shown in FIG. 6, in the subset table, information on one analysis file set is described per row. The information on the analysis file set includes the material of the sample to be analyzed, the preprocessing conditions of the sample, the type of the analyzer (device type) that measured the sample, the measurement conditions in the analyzer, and the analysis data.

As described above, a subset is an analysis file set group in which the analyzer type, the preprocessing conditions of the sample and the measurement conditions all match. The analysis file set group may include a plurality of analysis file sets per one material from the standpoint of ensuring the accuracy of analysis data, as described above. In the subset table, the plurality of these analysis file sets is listed with each row distinguished from the others. Therefore, the number of rows may differ among materials.

In the example shown in FIG. 6, the subset table shows an analysis file set group in which the analyzer type is a “GC-MS,” the sample preprocessing condition is “Pattern 1”, and the measurement condition is “Condition 1.” The analysis data in each analysis file set include chromatograms, mass spectra, and TICs acquired from a GC-MS.

The display data generation unit 32 generates data to display the subset table generated by the subset table generation unit 26 (see FIG. 6) on the display screen of the display unit 2. By referring to the subset table displayed on the display unit 2, it is possible for the user to visually confirm the details of each of the analysis data constituting the subset. Further, the user can grasp that there exist multiple analysis data in which the preprocessing conditions of the sample, data processing apparatus type, and measurement conditions all match, for one material. Further, the user can determine what standardization processing should be performed on the subset.

The standardization processing unit 28 performs standardization processing on multiple analysis data included in the subset table. This standardization processing puts the multiple analysis data into a form or easier comparison. Alternatively, quantitative values are acquired from each piece of analysis data using the same analysis technique. In the example in FIG. 6, the standardization processing unit 28 performs processing to calculate a peak area from a chromatogram and alignment processing to correct deviations in the retention times of a plurality of TICs.

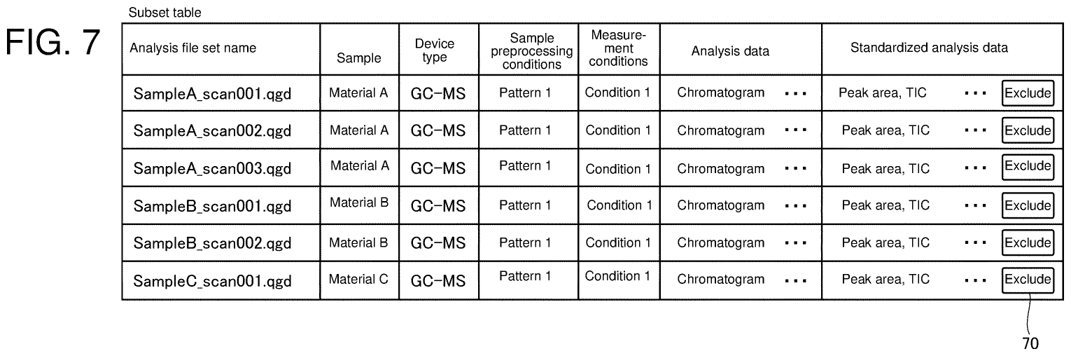

FIG. 7 is a diagram showing one example of a subset table to which standardization processing has been performed. The standardization processing unit 28 records the data generated by the standardization processing of the corresponding analysis data in each row of the subset table.

The user can determine whether there are any measurement errors in the analysis data or whether the standardized data contain outliers by referring to the details of the analysis data and the standardized data. In the case where it is determined that there are measurement errors and/or outliers, the user can exclude the analysis file set from the analysis file set group. This is because generating a feature table using a subset table including defective data may lead to defects in later machine learning.

Specifically, the subset table includes an icon 70 for instructing to “exclude” from the analysis file set group for each analysis file set. In the case where it is determined that any of the analysis data and features are defective, the user may exclude the analysis file set from the subset table by clicking on the icon 70 corresponding to the analysis file set including the data. In response to the user operation, the standardization processing unit 28 excludes the analysis file set specified by the user from the subset table.

Although not shown in the figure, instead of the configuration to exclude an analysis file set, it may be configured to exclude only the defective data from the analysis file set.

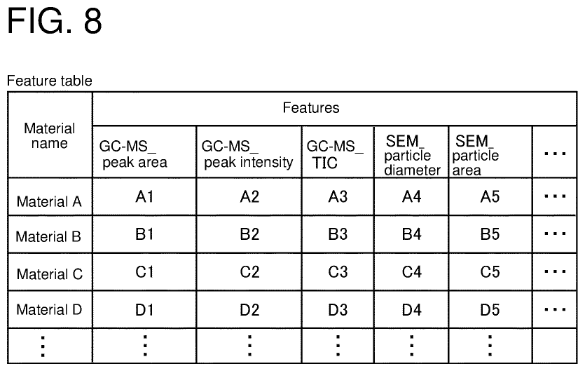

The representative value calculation unit 30 uses the subset table generated by the standardization processing unit 28 to generate a feature table recording feature values. In this specification, the “feature table” is a table format that summarizes features used for analyses when performing statistical and/or AI analyses of the plurality of analysis file sets. FIG. 8 is a diagram showing one example of a feature table. As shown in FIG. 8, the feature table records, for each material, multiple types of features extracted from the plurality of analysis file sets belonging to the material. The feature table can record, in addition to the features, the physical properties of the sample and the calculated value acquired by performing arithmetic processing on at least one of the features.

The representative value calculation unit 30 calculates representative values of the data for each material, for each subset table. In the case where one subset table contains multiple analysis data of the same material, the representative value calculation unit 30 calculates representative values of multiple data generated by the standardization processing of multiple analysis data. The representative value is a statistic that is a numerical value that summarizes a plurality of features. This statistic is also referred to as a summary statistic. Representative values include, for example, a mean value, a median value, a mode, a maximum value, a minimum value, a standard deviation, a variance, a skewness, a kurtosis, and so on. Among these statistics, the mean value, the median value, the mode, the maximum value, and the minimum value correspond to a representative value that represents the entirety of the features for each material. A standard deviation, a variance, a skewness, and a kurtosis correspond to a scatter that represents the dispersion of the features in each group.

Note that the types of representative values can be configured such that the user sets the type of the representative value by referring to the subset table and inputs it to the data processing apparatus 1. Alternatively, it can be configured such that the user specifies the type of the representative value in advance and gives it to the data processing apparatus 1, depending on the type of standardized data.

The representative value calculation unit 30 then records the calculated representative values in the feature table. The generated feature table is stored in the feature DB 15B.

The analysis unit 34 performs an AI analysis, a statistical analysis, etc., using the feature table stored in the feature DB 15B. The method of machine learning in the analysis unit 34 is not particularly limited, and for example, known machine learning, such as, e.g., neural networks (NN: Neural Network) and support vector machines (SVM: Support Vector Machine) can be used.

### <Operation of Data Processing Apparatus 1>

Next, the processing performed by the data processing apparatus 1 will be described.

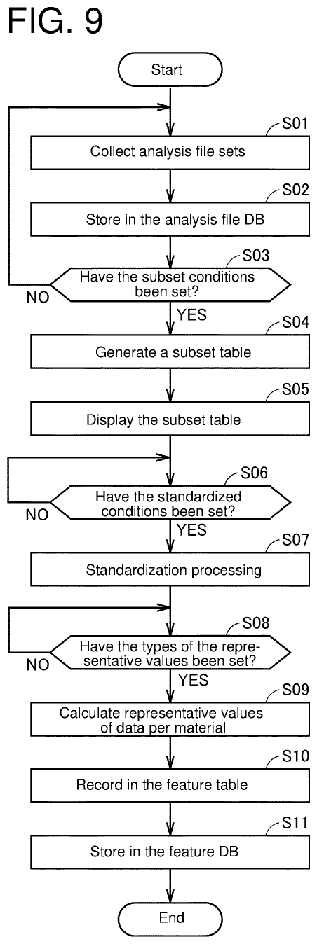

FIG. 9 is a flowchart for explaining the processing steps performed by the data processing apparatus 1.

As shown in FIG. 9, first, in Step (hereafter simply “S”) 01, the data processing apparatus 1 collects analysis file sets transmitted from the information processing apparatuses 6 of the plurality of analyzers 4 via the communication I/F 13.

In S02, the data processing apparatus 1 stores the plurality of collected analysis file sets in the analysis file DB 15A (see FIG. 4). In S02, the data processing apparatus 1 can store the sample information (sample identification information and recipe data) and the sample physical property data in a linked manner for each analysis file set. As shown in FIG. 4, in the analysis file DB 15A, the plurality of analysis file sets is sorted and stored for each material in one collection.

In S03, the data processing apparatus 1 determines whether the conditions regarding the subset have been set, based on the user's input operation to the operation unit 3. In a scene of generating a feature table (see FIG. 8) to be used for machine learning, the user can set the conditions for extracting an analysis file set that constitutes a subset from one collection by operating the UI screen displayed on the display unit 2 with the operation unit 3. Specifically, the user can set preprocessing conditions of a sample, instrument types, and measurement conditions.

Once the conditions for the subset are set (YES in S03), the data processing apparatus 1 generates subset tables from the plurality of analysis file sets belonging to one collection, in S04. In S04, the data processing apparatus 1 extracts the analysis file set in which the apparatus type, the sample preprocessing conditions, and the measurement conditions all match, from the plurality of analysis file sets and forms subsets. And, the data processing apparatus 1 generates a subset table (see FIG. 6) with one analysis file set per line.

In S05, the data processing apparatus 1 displays the generated subset table on the display unit 2.

In S06, the data processing apparatus 1 determines whether the conditions for the standardization processing have been set, based on the user's input operation to the operation unit 3. The user can set what kind of standardization processing will be performed on the subset table by operating the UI screen displayed on the display unit 2 using the operation unit 3. For example, it is possible to set, for example, whether alignment processing of waveform data should be performed and what quantitative values should be acquired from the waveform data.

Once the conditions for the standardization processing are set (YES in S06), the data processing apparatus 1 performs standardization processing in accordance with the set conditions on the multiple analysis data included in the subset table in S07. The data processing apparatus 1 records the data generated by the standardization processing of the corresponding analysis data in each row of the subset table displayed in the display unit 2 (see FIG. 7).

In S08, the data processing apparatus 1 determines whether the type of the representative value has been set, based on the user's input operation to the operation unit 3. The user can set the type of the representative value to be calculated from the subset table by operating the UI screen displayed on the display unit 2 using the operation unit 3. or example, it is possible to set an average or a median value of the data for each material.

Once the type of the representative value is set (YES in S08), the data processing apparatus 1 calculates the representative value of the data for each material in the subset table in S09. In S09, the data processing apparatus 1 calculates the representative value of the multiple data generated by the standardization processing for each material.

Note that as explained with reference to FIG. 7, the data processing apparatus 1 can exclude at least one analysis file set from the subset table according to the user's input operation to the operation unit 3. In the case where at least one analysis file set is excluded, the data processing apparatus 1 calculates the representative value for each material from the remaining analysis file sets in the subset table.

In S10, the data processing apparatus 1 records the calculated representative value for each material in the feature table (see FIG. 8) as the feature value for the material.

In S11, the data processing apparatus 1 stores the generated feature table in the feature DB 15B. The stored feature tables are used for machine learning.

### Effects of This Embodiment

As described above, the data processing apparatus 1 according to this embodiment is configured to generate a subset table from the plurality of analysis file sets collected from the multiple types of analyzers 4 as a preliminary step to generate a feature table to be used for machine learning.

Specifically, the data processing apparatus 1 extracts an analysis data group to be subjected to the same standardization processing to compare or summarize the data, from a plurality of analysis file sets belonging to one collection and forms one subset. The subset is constituted by an analysis file set group in which the sample preprocessing conditions, the analyzer type, and the measurement conditions all match. The data processing apparatus 1 generates a subset table with information on one analysis file set per line.

This eliminates the need for the user to search for analysis file sets that meet the above conditions from the analysis file DB 15A storing a wide variety of analysis file sets.

In addition, since the subset table includes information on one analysis file set per line regardless of material dissimilarity, the same standardization processing can be uniformly performed on the analysis data in the analysis file set group constituting the subset. Then, by obtaining the representative value for each material from the subset table with standardization processing, it is possible to efficiently generate a feature table that records the features for each material.

In addition, since the subset table contains the analysis data for each analysis file set and the standardized data for the analysis data, in the case where some analysis file sets are determined to be defective in data, the user may exclude such data or the analysis file set containing such data from the subset table. In this case, standardization processing is performed on the subset table from which the data or analysis file set has been excluded to calculate representative values for each material. Therefore, it is possible to prevent a feature table from being generated using subset tables with defective data.

### ASPECTS

It would be understood by those skilled in the art that the exemplary embodiments described above are specific examples of the following aspects.

### (Item 1)

A data processing method according to one aspect of the present invention comprising:
a step for a computer to collect analysis file sets from multiple types of analyzers, the analysis file sets each including analysis data by the analyzer;
a step for the computer to store the plurality of collected analysis file sets in a database with the analysis file sets sorted into corresponding collections for each material; and
a step for the computer to form a subset by extracting an analysis file set in which a type of the analyzer, preprocessing conditions of a sample measured by the analyzer, and measurement conditions of the sample all match, from the collection.

According to the data processing method as recited in the above-described Item 1, it is possible to easily extract and summarize an analysis file set group for which the same standardization processing is to be performed to compare or summarize its analysis data, from a database storing a wide variety of analysis file sets collected from a plurality of analysis apparatuses.

### (Item 2)

The data processing method as recited in the above-described Item 1, further comprising:
a step for the computer to perform standardization processing of the analysis data on an analysis file set group constituting the subset;
a step for the computer to calculate representative values of the analysis data for each material from the analysis file set group that has undergone the standardization processing; and
a step for the computer to record the calculated representative values of the analysis data for each material in a feature table, as features of the material.

According to the data processing method as recited in the above-described Item 2, it is possible to uniformly perform the same standardization processing on the analysis data in the analysis file set group constituting the subset. Then, by obtaining the representative value for each material from the subset table with standardization processing, it is possible to efficiently generate a feature table recording the features for each material.

### (Item 3)

The data processing method as recited in the above-described Item 2,
wherein the step of performing the standardization processing includes a step of transforming the analysis data in the analysis file set group into a form for easier comparison or summarization.

According to the data processing method as recited in the above-described Item 3, it is possible to calculate representative values for each appropriate material from the subset.

### (Item 4)

The data processing method as recited in any one of the above-described Items 1 to 3, further comprising:
a step for the computer to present the analysis data included in an analysis file set group constituting the subset to a user.

According to the data processing method as recited in the above-described Item 4, the user can visually confirm the details of each analysis data constituting the subset by referring to the presented subset. Further, the user can grasp that there exist multiple analysis data in which the sample preprocessing conditions, data processing apparatus type, and measurement conditions all match, for one material. Further, the user can determine what standardization processing should be performed on the subset.

### (Item 5)

The data processing method as recited in claim 4,
wherein the step of presenting to the user includes a step of presenting a subset table to the user, the subset table describing the analysis data of one analysis file set on one row.

According to the data processing method as recited in the above-described Item 5, the subset table contains information on one analysis file set per line regardless of material dissimilarity, so that the same standardization processing can be uniformly performed on the analysis data in the analysis file set group constituting the subset.

### (Item 6)

The data processing method as recited in any one of the above-described Items 2 to 5, further comprising:
a step for the computer to exclude at least one analysis file set from the subset in response to a user instruction.

The step of calculating the representative values includes a step of calculating the representative values of the analysis data for each material from the subset from which the at least one analysis file set has been excluded.

According to the data processing method as recited in the above-described Item 6, it is possible to prevent a feature table from being generated based on a subset with defective data.

### (Item 7)

In the data processing method as recited in any one of the above-described Items 1 to 6,
in the step of forming the subset, the subset is formed over material differences within one collection, and an analysis file set belonging to another collection is not included in the subset.

According to the data processing method as recited in the above-described Item 7, it is possible to prevent subsets from being formed beyond a collection of materials that are to be used for AI analyses or statistical analyses.

### (Item 8)

A data processing apparatus as recited in the above-described Item 8 is a data processing apparatus capable of communicating with multiple types of analyzers, the data processing apparatus comprising:
a processor; and
a memory configured to store programs to be executed by the processor.

The processor is configured, according to the programs, to collect analysis file sets from the multiple types of analyzers, the analysis file sets each including analysis data by the analyzer.

The processor is configured to store the plurality of collected analysis file sets in a database with the analysis file sets sorted into corresponding collections for each material.

The processor is configured to form a subset by extracting an analysis file set in which a type of the analyzer, preprocessing conditions of a sample measured by the analyzer, and measurement conditions of the sample all match, from the collection.

According to the data processing method as recited in the above-described Item 8, it is possible to easily extract and summarize an analysis file set group for which the same standardization processing is to be performed to compare or summarize its analysis data from a database storing a wide variety of analysis file sets collected from a plurality of analysis apparatuses.

### (Item 9)

A non-transitory computer-readable storage medium as recited in the above-described Item 9 stores programs.

The programs make a computer execute
a step of collecting analysis file sets from multiple types of analyzers, the analysis file sets each including analysis data by the analyzer,
a step of storing the plurality of collected analysis file sets in a database with the analysis file sets sorted into corresponding collections for each material, and
a step of forming a subset by extracting an analysis file set in which a type of the analyzer, preprocessing conditions of a sample measured by the analyzer, and measurement conditions of the sample all match, from the collection.

According to the data processing method as recited in the above-described Item 9, it is possible to easily extract and summarize an analysis file set group for which the same standardization processing is to be performed to compare or summarize its analysis data from a database storing a wide variety of analysis file sets collected from a plurality of analysis apparatuses.

Further, it should be noted that in the embodiment and modifications, it is planned from the beginning of the application to combine the configurations described in the embodiments as appropriate, including combinations not mentioned in the specification, to the extent that no inconvenience or inconsistency arises.

Although some embodiments of the present invention have been described, the embodiments disclosed here should be considered in all respects illustrative and not restrictive. It should be noted that the scope of the invention is indicated by claims and is intended to include all modifications within the meaning and scope of the claims and equivalents.

1. A data processing method comprising:
a step for a computer to collect analysis file sets from multiple types of analyzers, the analysis file sets each including analysis data by the analyzer;
a step for the computer to store the plurality of collected analysis file sets in a database with the analysis file sets sorted into corresponding collections for each material; and
a step for the computer to form a subset by extracting an analysis file set in which a type of the analyzer, preprocessing conditions of a sample measured by the analyzer, and measurement conditions of the sample all match, from the collection.

2. The data processing method as recited in claim 1, further comprising:
a step for the computer to perform standardization processing of the analysis data on an analysis file set group constituting the subset;
a step for the computer to calculate representative values of the analysis data for each material from the analysis file set group that has undergone the standardization processing; and
a step for the computer to record the calculated representative values of the analysis data for each material in a feature table, as features of the material.

3. The data processing method as recited in claim 2,
wherein the step of performing the standardization processing includes a step of transforming the analysis data in the analysis file set group into a form for easier comparison or summarization.

4. The data processing method as recited in claim 1, further comprising:
a step for the computer to present the analysis data included in an analysis file set group constituting the subset to a user.

5. The data processing method as recited in claim 4,
wherein the step of presenting to the user includes a step of presenting a subset table to the user, the subset table describing the analysis data of one analysis file set on one row.

6. The data processing method as recited in claim 2, further comprising:
a step for the computer to exclude at least one analysis file set from the subset in response to a user instruction,
wherein the step of calculating the representative values includes a step of calculating the representative values of the analysis data for each material from the subset from which the at least one analysis file set has been excluded.

7. The data processing method as recited in claim 1,
wherein in the step of forming the subset, the subset is formed over material differences within one collection, and an analysis file set belonging to another collection is not included in the subset.

8. A data processing apparatus capable of communicating with multiple types of analyzers, comprising:
a processor; and
a memory configured to store programs to be executed by the processor,
wherein the processor is configured, according to the programs, to
collect analysis file sets from the multiple types of analyzers, the analysis file sets each including analysis data by the analyzer,
store the plurality of collected analysis file sets in a database with the analysis file sets sorted into corresponding collections for each material, and
form a subset by extracting an analysis file set in which a type of the analyzer, preprocessing conditions of a sample measured by the analyzer, and measurement conditions of the sample all match, from the collection.

9. A non-transitory computer-readable storage medium storing programs,
wherein the programs make a computer execute
a step of collecting analysis file sets from multiple types of analyzers, the analysis file sets each including analysis data by the analyzer,
a step of storing the plurality of collected analysis file sets in a database with the analysis file sets sorted into corresponding collections for each material, and
a step of forming a subset by extracting an analysis file set in which a type of the analyzer, preprocessing conditions of a sample measured by the analyzer, and measurement conditions of the sample all match, from the collection.
